(19) United States
(12) Patent Application Publication
(10) Pub. No.: US 2010/0111922 A1
(43) Pub. Date: May 6, 2010

(54) Animal model and use of 17beta-hydroxysteroid dehydrogenase type 7 in the diagnosis of anencephaly

(76) Inventors: Guela Gibori, Chicago, IL (US); Aurora Shehu, Chicago, IL (US)

Correspondence Address: LICATA & TYRRELL P.C., 66 E. MAIN STREET, MARLTON, NJ, 08053, US

(21) Appl. No.: 12/607,255

(22) Filed: Oct. 28, 2009

Related U.S. Application Data
(60) Provisional application No. 61/110,662, filed on Nov. 3, 2008.
(60) Provisional application No. 61/139,728, filed on Dec. 22, 2008.

(51) Int. Cl. A61K 38/44; A01K 67/027; C12N 5/10; C12Q 1/68; A61K 31/7088; C07H 21/04; A61P 25/00; G01N 33/48; C12N 9/02

(52) U.S. Cl. 424/94.4; 800/13; 435/325; 435/6; 514/44 R; 536/23.2; 800/3; 435/189

(57) ABSTRACT

The present invention features a non-human animal in which is deficient in the expression of endogenous 17β Hydroxysteroid Dehydrogenases Type 7 and use of the same in screening methods for agents that prevent or treat anencephaly. The present invention also provides a method for diagnosing and treating.

This application claims benefit of priority of U.S. 61/110,662, filed Nov. 3, 2008 and 61/139,728, filed Dec. 22, 2008, the contents of which are incorporated herein by reference in their entireties.

This invention was made with government support under Grant Nos. HD011119 and HD012356 awarded by the National Institutes of Health. The government has certain rights in the invention.

### BACKGROUND OF THE INVENTION

The synthesis of estradiol in the ovary requires the expression of two enzymes, P450 aromatase (CYP19) and 17β hydroxysteroid dehydrogenases (17βHSD). Whereas CYP19 in rodents is found both in the follicle and corpus luteum, two different 17βHSDs are expressed in a cell specific manner in the ovary (Stocco, et al. (2006) Endocr. Rev. 28:117-149). In the follicle, specifically the granulosa cells, 17β hydroxysteroid dehydrogenase type 1 (HSD17B1) converts estrone, generated by the conversion of androstendione by CYP19, to estradiol (Stocco, et al. (2006) supra; Akinola, et al. (1998) Gene 208:229-238). HSD17B1 also catalyzes the conversion of androstenedione to testosterone (Ghersevich, et al. (1994) Endocrinology 135:1477-1487), which can be aromatized to estradiol directly. HSD17B1 was the first to be identified in the follicle characterized and crystallized (Akinola, et al. (1998) supra; Luu-The, et al. (1989) Mol. Endocrinol. 3:1301-1309; Peltoketo, et al. (1988) FEBS Lett. 239:73-77; Ghosh, et al. (1995) Structure 3:503-513; Alho-Richmond, et al. (2006) Mol. Cell Endocrinol. 248:208-213), yet no expression of this enzyme could be detected in the corpus luteum. Despite the fact that the rodent corpus luteum was shown to express CYP19 and able to synthesize estradiol (Stocco, et al. (2006) supra; Hickey, et al. (1989) Endocrinology 125:1673-1682), the luteal 17βHSD responsible for converting estrone to estradiol and/or androstenedione to testosterone was not readily identified. However, a protein in the rat corpus luteum (McLean, et al. (1990) Endocrinology 126:1796-1805) was subsequently identified as a novel 17βHSD enzyme (Nokelainen, et al. (1998) Mol. Endocrinol. 12:1048-1059; Risk, et al. (1999) Biol. Reprod. Suppl. 60:162). This enzyme, which has little homology to HSD17B1, was originally identified as a 32-kDa protein abundantly expressed in the large luteal cells of the corpus luteum (McLean, et al. (1990) supra). Subsequently, the cDNA was cloned (Duan, et al. (1996) J. Biol. Chem. 271:15602-15607), specific antibodies were developed (Parmer, et al. (1992) Endocrinology 131:2213-2221) and the promoter region of this gene was isolated and characterized (Risk, et al. (2005) Endocrinology 146:2807-2816). This protein was found to be phosphorylated on tyrosine and to associate with the intracellular domain of the short form of the prolactin receptor (Duan, et al. (1996) supra). Therefore, this protein was originally named PRAP (prolactin receptor-associated protein). Once it was established that PRAP was a novel 17βHSD (Nokelainen, et al. (1998) supra; Risk, et al. (1999) supra) it was consequently renamed PRAP/17βHSD-7 or HSD17B7. In contrast to HSD17B1 (Stocco, et al. (2006) supra; Akinola, et al. (1998) supra; Ghersevich, et al. (1994) supra), HSD17B7 converts estrone to estradiol but not androstenedione to testosterone (Nokelainen, et al. (1998) supra). It is found in the corpus luteum of every species investigated, including ruminants and humans (Parmer, et al. (1992) supra), revealing the possibility that the corpus luteum has a universal capacity to convert estrone to estradiol. After luteinization, HSD17B1 disappears from the follicles and the luteal HSD17B7 becomes the enzyme responsible for the production of estradiol, especially during pregnancy (Stocco, et al. (2006) supra; Parmer, et al. (1992) supra; Peltoketo, et al. (1999) J. Steroid Biochem. Mol. Biol. 69:431-43915). This luteal cell-derived estradiol is shown to play a key role in the hypertrophy, vascularization and progesterone production by the corpus luteum in rodents and is considered to be, together with prolactin, a tropic hormone essential for corpus luteum survival and steroiodogenesis (Stocco, et al. (2006) supra; Risk & Gibori (2001) In: Horseman N D, ed. Prolactin. Dordrecht: Kluwer Academic Publisher, 265-295; Bowen-Shauver & Gibori (2004) In: Adashi E Y, Leung P C K eds. The Ovary, NY, Raven Press, 201-232).

HSD17B7 has also been described as possessing dual enzymatic activity. In addition to conversion of estrone to estradiol, it participates in postsqualene cholesterol biosynthesis and converts zymosterone to zymosterol in vitro (Breitling, et al. (2001) Mol. Cell Endocrinol. 171:199-204; Marijanovic, et al. (2003) Mol. Endocrinol. 17:1715-1725). In this regard, US Patent Application No. 20080261274 teaches the use of the HSD17b7 gene for complementation to restore cholesterol independence to NS0 cells. NS0 is a mammalian cell line widely used in recombinant genetic engineering. NS1 cells and derivatives, including NS0 cells, are cholesterol auxotrophs.

In addition, WO 2008/009856 provides an in vitro method of screening for candidate compounds for the preventive or curative treatment of acne based upon the ability of the compound to modulate expression or activity of HSD17b7.

### SUMMARY OF THE INVENTION

The present invention features a knockout transgenic non-human animal whose genome includes a deletion in the endogenous 17β Hydroxysteroid Dehydrogenases Type 7 gene, wherein said animal exhibits one or more signs or symptoms of neuronal tube defects indicative of anencephaly. A method of screening for a prophylactic or therapeutic agent for anencephaly using the knockout transgenic non-human animal is also provided as is a method for using such an agent in the prevention or treatment of anencephaly. In particular embodiments, the agent is 17β Hydroxysteroid Dehydrogenases Type 7 protein or a nucleic acid encoding the same.

The present invention also features a method of diagnosing anencephaly by determining whether a subject has a mutation, deletion or insertion in the gene encoding 17β Hydroxysteroid Dehydrogenases Type 7 (HSD17B7) or has a decrease in the expression or activity of HSD17B7 as compared to a control, wherein said mutation, deletion or decrease in expression or activity of HSD17B7 is indicative of anencephaly.

### BRIEF DESCRIPTION OF THE DRAWING

FIG. 1 depicts the targeted disruption of the HSD17B7 gene. Shown is a partial restriction map of the mouse Hsd17b7 genomic locus, the targeting vector, and the structure of the locus following recombination, as well as the targeting construct contains 5′ flanking coding region upstream of Hsd17b7 and 3′ exons 5 through 7. Homologous recombination within the genomic sequence introduces the neo gene and eliminates exons 1 through 4. Restriction endonuclease sites used for cloning are shown.

### DETAILED DESCRIPTION OF THE INVENTION

The present invention features a knockout non-human transgenic animal lacking an endogenous 17β Hydroxysteroid Dehydrogenases Type 7 (HSD17B7) gene. This enzyme with dual activity is involved in both estradiol and cholesterol biosynthesis has now been shown to play a significant role in fetal development. In particular, non-human transgenic animals lacking an endogenous HSD17B7 gene exhibit one or more common signs or symptoms associated with anencephaly, e.g., neural tube and cardiovascular defects. Accordingly, the present invention further features a method for diagnosing anencephaly based upon determination of the expression level or activity of HSD17B7 and use of HSD17B7 as a therapeutic target for anencephaly in humans.

Anencephaly is a cephalic disorder that results from a neural tube defect that occurs when the cephalic (head) end of the neural tube fails to close, usually between the 23rd and 26th day of pregnancy in humans, resulting in the absence of a major portion of the brain, skull, and scalp. Children with this disorder are born can exhibit one or more of the following symptoms: absence of bony covering over the back of the head; missing bones around the front and sides of the head; folding of the ears, cleft palate, a condition in which the roof of the child's mouth does not completely close, leaving an opening that can extend into the nasal cavity; congenital heart defects; and minimal basic reflexes.

Fetal cholesterol biosynthesis rather than cholesterol transfer from maternal lipoproteins appears to be the main mechanism for satisfying fetal requirements. From E10, the blood-brain barrier is formed in the rodents and the brain rapidly becomes the source of almost all of its own cholesterol. In the HSD17B7 null embryos of this invention, E10 is the stage where severe defects in brain formation are most apparent resulting in distress; lack vascularization; a drastic reduction in size of the forebrain and the fourth ventricle in the hindbrain region, and a marked underdevelopment and reduced size and volume in the forebrain and midbrain. In addition, null embryos also had a heart defect with abnormal accumulation of fluid in the pericardial cavity. In this regard, the non-human transgenic animal of this invention exhibits one or more common signs or symptoms of neuronal tube defects indicative of anencephaly in humans.

The knockout transgenic non-human animal of the invention is an animal created by genetic engineering. Non-human animals of the invention can be generated using any conventional method including, but not limited to, the use of embryonic stem cells (hereinafter, referred to as “ES cells”). See, for example, in U.S. Pat. Nos. 4,736,866 and 4,870,009. Significant developments have been made in the use of ES cells to generate transgenic animal including homologous recombination of ES cells (Thomas & Capecchi (1987) Cell 51:503), germ line transmission of ES cell characteristics, and creation of gene-deficient or knockout mice. Indeed, knockout animals have been described for the HPRT gene (Hooper, et al. (1987) Nature 326:292; Knehn, et al. (1987) Nature 326:295); p53 gene (Donehower, et al. (1992) Nature 356:215); β2 microglobulin gene (Zijlstra, et al. (1990) Nature 344:742); RAG-2 (V(D)J recombination activation) gene (Sinkai, et al. (1992) Cell 68:855); MHC class II (Glimcher, et al. (1991) Science 253:1417; Cosgrove, et al. (1991) Cell 66:1051); int-1 gene (MacMahon, et al. (1990) Cell 62:1073); and src gene (Soriano, et al. (1991) Cell 64:693, to name a few, such that the methods for generating knockout non-human animals is routine in the art.

A “knockout transgenic non-human animal whose genome includes a deletion in the endogenous HSD17B7 gene” means an animal, in particular a mammal, in which the HSD17B7 gene, through introduction of an artificial mutation to the endogenous HSD17B7 gene, exhibits a substantial or complete loss in the expression of HSD17B7 protein. In this regard, a “knockout” is an alteration in the nucleic acid sequence that reduces the biological activity of the HSD17B7 by at least 80% compared to the unaltered gene. The alteration may be an insertion, deletion, frameshift mutation, or missense mutation. Preferably, the alteration is an insertion or deletion, or is a frameshift mutation that creates a stop codon.

For the purpose of creating a transgenic animal (preferably, non-human mammal), an ES cell can be employed wherein the HSD17B7 gene is inactivated (hereinafter, referred to as a “HSD17B7 gene-inactivated ES cell” or “knockout ES cell”). The knockout ES cell is produced by preparing and transfecting/delivering a targeting vector which has a DNA sequence constructed so that the exon function of HSD17B7 gene is destroyed, e.g., by insertion of a drug-resistance gene (e.g. neomycin resistance gene, hygromycin resistance gene, or zeocin resistance gene) or a reporter gene (e.g., lacZ, chloramphenicol acetyltransferase gene, β-glucuronidase gene, luciferase gene, aequorin gene, thaumarin gene, or green fluorescent protein gene) into its exon region, or a DNA sequence for terminating gene transcription (e.g., poly A addition signal) in an intron region between exons, thereby inhibiting synthesis of complete messenger RNA transcription, resulting in lack of HSD17B7 protein production. When destruction of the exon function by insertion of a reporter gene is intended, it is preferable to insert the reporter gene so that it is expressed under the control of the HSD17B7 promoter.

As known in the art, the HSD17B7 gene being targeted will be dependent upon the animal employed. In this regard the HSD17B7 gene can encode a mouse (GENBANK Accession No. NP_{—}034606; SEQ ID NO:1), rat (GENBANK Accession No. NP_{—}058931; SEQ ID NO:2), dog (GENBANK Accession No. XP_{—}851698), or homolog of the human (GENBANK Accession No. NP_{—}057455) HSD17B7 protein.

The targeting vector can be a plasmid derived from Escherichia coli (e.g., pBR322, pBR325, pUC12, and pUC13), Bacillus subtilis (e.g., pUB110, pTP5 and pC194), yeast (e.g., pSH19 and pSH15); a bacteriophage such as λ-phage; or an animal viruses such as a retrovirus (e.g., Moloney leukemia virus), vaccinia virus or adenovirus vector, baculovirus, bovine papilloma virus, virus from herpes virus group, or Epstein-Barr virus.

Subsequently, the above-described targeting vector is introduced into chromosomes of ES cells by homologous recombination. The resultant ES cells are analyzed either by Southern hybridization using as a probe a DNA sequence on the HSD17B7 gene outside of the targeting vector or in the vicinity thereof; or analyzed by PCR using as primers a DNA sequence on the targeting vector and a DNA sequence in the vicinity of the HSD17B7 gene other than the sequence used for preparing the targeting vector. Thus, the knockout ES cell of the invention can be selected.

The HSD17B7 gene is then knocked out in vivo by introducing the targeting vector described above into non-human animal ES cells or non-human animal blastocyst according to conventional methods, e.g., electroporation, microinjection, the calcium phosphate method, lipofection, agglutination, or the particle gun method, or the DEAE-dextran method, to thereby homologously recombine the inactivated HSD17B7 gene sequence on the targeting vector with the HSD17B7 gene on the chromosome of the non-human animal ES cells or non-human animal blastocyst.

Non-human animals, in particular mammals, which can be used as sources for ES cells of this invention include, bovine, pig, sheep, goat, rabbit, dog, cat, guinea pig, hamster, mouse, rat, etc. From the viewpoint of construction of disease animal models, rodents are of particular use because they have relatively short ontogenesis and life cycles and can be easily propagated. In particular embodiments, a mouse (e.g., pure strains such as C57B/6, DBA2, etc. and hybrid strains such as B6C3F₁, BDF₁, B6D2F₁, BALB/c, ICR, etc.) or rat (e.g., Wistar, SD, etc.) is employed. Established ES cell lines can also be used. Such cells include, e.g., EK cells (Evans & Kaufman (1981) Nature 292:154), ES-D3 cells derived from 129/Sv+c/+p mice (Doetschman (1981) J. Embryol. Exp. Morph. 87:27), CCE cells (Robertson (1986) Nature 323:445), BL/6III cells derived from C57/BL6 mice, and E14TG2a ES cells derived from 129/OLA mice.

When a part of the germ cells of the chimeric animal has the mutated HSD17B7 locus, it is possible to mate such a chimeric individual with a normal individual to thereby obtain offspring individuals, and then select those individuals in which every tissue is composed of cells having the artificially mutated HSD17B7 locus, for example, by judging their coat colors. Selected individuals are usually HSD17B7 hetero-deficient individuals. HSD17B7 homo-deficient individuals can be obtained from offspring individuals produced by mating these HSD17B7 hetero-deficient individuals with each other.

Mammalian ES cells, in which the HSD17B7 gene is inactivated, are useful in creating a non-human animal deficient in expression of HSD17B7 gene products (i.e., mRNA or protein). The resulting non-human animal deficient in expression of HSD17B7 gene finds application as an anencephaly model which can be used to study the development of anencephaly as well as identify therapies for the prevention or treatment (e.g., lessening of symptoms) of anencephaly. Moreover, the transgenic animal of this invention can mated with other disease model animals to study the influence of HSD17B7 deficiency in other diseases.

In so far as fetal cholesterol biosynthesis has been identified as a significant factor in anencephaly and HSD17B7 is a key enzyme of this pathway, it is contemplated that mutations in other key enzymes in this pathway will also contribute to anencephaly. These enzymes include, HMG-CoA Synthase, HMG-CoA Reductase, Mevalonate Kinase, Phosphomevalonate Kinase, Pyrophosphomevolanate Decarboxylase, Isopentenyl Pyrophosphate Isomerase, Prenyl Transferase (Farnesyl Pyrophosphate Synthase), Squalene Synthase, Squalene epoxidase and Squalene Oxidocyclase. In this regard, transgenic knockouts animals deficient in one or more of the genes encoding these enzymes are also expected to exhibit one or more signs associated with anencephaly.

In accordance with another feature of this invention, the instant transgenic animals or tissues or cells or cell lines derived from transgenic animal thereof are employed in a screening method for identifying agents useful in the detection, prevention or treatment of anencephaly. In particular embodiments, embryos which are null for HSD17B7 can be used for the development of cell lines for use as diagnostic and analytical tools, e.g., drug and gene therapy. This method involves contacting a cell, cell line, tissue or non-human transgenic animal of the invention with a test agent and determining whether the test agent detects, corrects, prevents, or delays anencephaly when compared with non-treated control animals. Prophylactic or therapeutic effects of the test agent can be tested by using, as indicators, changes in individual organs, tissues, cells or symptoms of anencephaly of the test animals. By way of illustrating the instant screening method, a test animal (i.e., a HSD17B7 deficient non-human animal) is administered directly a test compound in utero or indirectly through the pregnant mother, and changes in nervous system development are measured during gestational period. In general, agents for prevention or treatment of anencephaly will function by altering the amount or level of HSD17B7 or overcome a HSD17B7 deficiency thereby lessening or preventing at least one or symptom of anencephaly. Any agent, including a cholesterol-derived compound, which delays or prevents anencephaly can be used in the treatment of anencephaly in humans.

Examples of test agents which can be screened include peptides, proteins, non-peptidic compounds, lipids or sterols or cholesterol-derived compounds, nucleic acids, synthetic compounds, fermentation products, cell extracts, plant extracts, and animal tissue extracts. These compounds may be novel compounds or known compounds. Numerous methods are available for generating random or directed synthesis (e.g., semi-synthesis or total synthesis) of any number of chemical compounds, including, but not limited to, saccharide-, lipid-, peptide-, sterol- and nucleic acid-based compounds. Libraries of genomic DNA or cDNA may be generated by standard techniques and are also commercially available (Clontech Laboratories Inc., Palo Alto, Calif.). Nucleic acid libraries used to screen for compounds that alter HSD17B7 gene expression or HSD17B7 protein activity are not limited to the species from which the HSD17B7 gene or protein is derived. For example, a Xenopus cDNA may be found to encode a protein that alters human HSD17B7 gene expression or alters human HSD17B7 protein activity. However, in particular embodiments, HSD17B7 protein, or DNA encoding the HSD17B7 protein (e.g., the HSD17B7 gene) are screened for their ability to prevent or delay the onset of anencephaly.

Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, USA (Cambridge, Mass.).

In addition, byproducts or enzymes of cholesterol biosynthesis can be screened. These include, (e.g., Mevalonate, Pyrophosphmevalonate, Isopentenyl pyrophosphate, Dimethylallyl pyrophosphate, Isopentenyl pyrophosphate, Geranyl pyrophosphate, Farnesyl pyrophosphate, Squalene, 2,3-oxidosqualene, Lanosterol, 7-dehydrocholesterol, and cholesterol.

For therapeutic use, an agent identified by the instant screen can be prepared in the form of tablets (sugar-coated, if necessary), capsules, elixirs, microcapsules, or the like; or in the form of i.v. solutions or injections such as aseptic solutions or suspensions in water or other pharmaceutically acceptable liquids. Such preparations may be produced by conventional methods, for example, by mixing the agent with physiologically acceptable carriers, flavoring agents, excipients, vehicles, antiseptics, stabilizers, binders, etc. in unit dosage forms required for preparing generally approved pharmaceutical preparations. The amounts of active ingredients in these formulations are selected so that an appropriate dose within the specified range can be obtained. An appropriate dose can be dependent upon many factors including the stage of development, the nature of the agent being used and the condition of the patient. The determination of an appropriate dose is routine in the art and can be determined based upon data generated in animal models.

When used therapeutically, an agent of the invention (including sterols, or DNA encoding the HSD17B7 protein) can be administered to a subject having or predisposed to develop anencephaly to detect, prevent or delay the onset of at least one sign or symptom of anencephaly. In particular embodiments, the agent is administered in utero during the early stages of nervous system development to prevent at least one sign or symptom of anencephaly. In accordance with embodiments pertaining to the delivery of nucleic acids encoding HSD17B7, the invention embraces the use of any suitable delivery method including, naked DNA, retroviral, vaccinia virus, lentivirus and adenoviral delivery.

Subjects having, suspected of having, at predisposed to develop anencephaly, who might benefit from therapeutic treatment, can be identified by determining whether the subject has a mutation, deletion, or insertion in the gene encoding HSD17B7 or has a decrease in the expression or activity of HSD17B7. In this regard, the present invention also features a method for diagnosing anencephaly. The method involves obtaining a sample (e.g., amniotic fluid or fetal tissue) from a subject and determining the level of expression or activity of HSD17B7, or mutation, insertion or deletion of the HSD17B7 gene. Mutation, insertion or deletion can be detected by sequencing genomic DNA or mRNA collected from any fetal tissue, wherein said mutation, deletion, insertion results in amino acid substitution, truncation, and/or post-translation modification that decrease the expression and activity of HSD17B7. The effect of the mutation, insertion, or deletion could also be determined by measuring HSD17B7 expression (at the protein or RNA level) or activity, and may be deemed to be normal or abnormal (e.g., reduced or increased) by comparison to the level of a control individual. HSD17B7 protein activity level may be measured, for example, by the ability of the protein to convert estrone to estradiol or by the conversion of zymosterone to zymosterol.

Any abnormality in the HSD17B7 gene resulting in a reduction in the amount or activity of the HSD17B7 protein is an indication that the subject may have, or may be predisposed to develop, anencephaly.

The diagnostic method of this invention can be used alone or in combination with other routine diagnostic tests performed during pregnancy to evaluate a baby for anencephaly. Such additional tests include measuring alpha-fetoprotein, wherein abnormal levels of alpha-fetoprotein may indicate brain or spinal cord defects or chromosomal disorders; or ultrasound to view internal organs as they function, and to assess blood flow through various vessels. Indeed, screening for HSD17B7 can be included in commercially available genetic screening arrays provided to screen common birth or genetic defects in pregnant mothers.

In so far as it has also been demonstrated that HSD17B7 null embryos exhibit a cardiovascular defect, some embodiments of this invention also embrace the diagnosis and treatment of the cardiovascular disease using HSD17B7.

The invention is described in greater detail by the following non-limiting examples.

### Example 1

### Materials and Methods

Experimental Animals. Animals were kept under conditions of controlled light (0700-1900 hours) and temperature (22-24° C.) with free access to standard rodent chow and water. All experimental procedures were performed in accordance to the Guidelines of the National Institutes of Health Guide for the Care and Use of Laboratory Animals and were approved by the Institutional Animal Care and Use Committee.

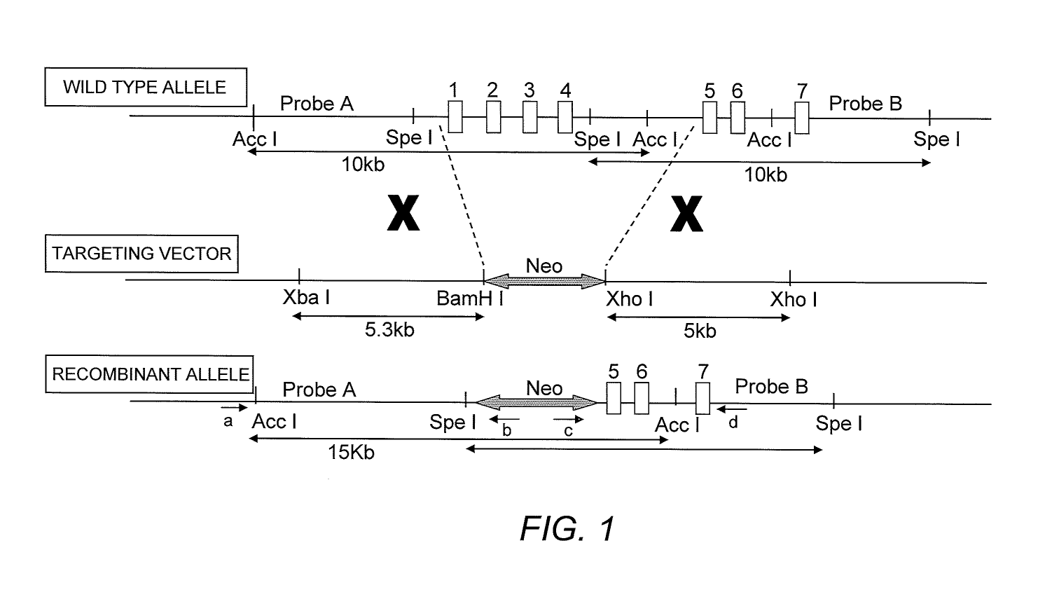

Generation of HSD17B7 Targeting Construct and Identification of Homologous Recombinant ES Cell Clones. Hsd17b7 was isolated by screening a mouse 129/SvJ genomic library (STRATAGENE, La Jolla, Calif.) using a probe containing mouse HSD17B7 exon 1 and exon 2. Twenty phage clones were selected and sequenced. Of the twenty clones selected, four clones contained fragments of different sizes encoding the 5′ upstream region and exons 1 through 7. These clones were subsequently subcloned into either pBS (STRATAGENE, La Jolla, Calif.) or pUC18 (INVITROGEN, Carlsbad, Calif.) vectors, re-sequenced and confirmed as part of the mouse Hsd17b7 gene by BLAST analysis. The HSD17B7 targeting vector was constructed by using a positive-negative selection vector, pOSDUPDEL (Gene Targeting Laboratory, The University of Iowa, Iowa City, Iowa). To generate the site of 5′ homologous recombination in the targeting vector, a 5.3-kb fragment containing 5′ upstream sequences of the Hsd17b7 gene was cloned into pOSDUPDEL at the XbaI-BamHI cloning site upstream of the neomycin phosphotransferase gene (neo) cassette. Orientation of this fragment was confirmed by restriction enzyme digestion and sequence analysis. Exons 1 through 4 were replaced by the neo cassette. To generate the 3′ homologous region, a 5.0-kb fragment, containing a part of intron 4 and exons 5 through 7, was isolated and inserted into pOSDUPDEL at the XhoI cloning site (FIG. 1). Orientation of this fragment was confirmed by restriction enzyme digestion and sequencing. In addition to neomycin, pOSDUPDEL contains a thymidine kinase cassette distal to the 5′ homologous region that is used as a secondary selection marker.

NotI-linearized targeting vector was introduced into CJ7 ES cells by electroporation. Positively transfected cells were selected in 0.5 mg/ml G418 and 0.2 μM ganciclovir. Two probes, probe A (0.7 kb) and probe B (0.8 kb), were used for Southern blot analysis of ES cells, following either AccI or SpeI digestion. To further confirm homologous recombination in ES cells, PCR was used to analyze the regions outside both the 5′ and 3′ homologous recombination sites extending into the neo cassette region (Primers (a-d) shown in third diagram of FIG. 1). In addition, PCR products were cloned into pGEM-Teasy (PROMEGA, Madison, Wis.) and sequenced, to confirm the sequence of the recombinant allele. Both PCR and Southern blot confirmed successful homologous recombination.

Following positive identification of homologous recombinant ES cell clones, recombinant ES cells were microinjected into C57BL/6 blastocysts and transplanted into uteri of pseudopregnant C57BL/6 mice. Chimeric males resulting from the microinjections were crossed to C57BL/6 females and agouti pups were screened for germ line transmission of the mutant allele. The genotypes from these matings and all subsequent matings were determined by both Southern blot and PCR analyses of tail DNA.

Genotyping of Mouse Embryos via Southern Blot Analysis. Genomic DNA was isolated and digested overnight with either AccI or SpeI. Samples were run on a 0.7% agarose gel and blotted on HYBOND-N+ (GE Healthcare, UK). The blots were prehybridized for 4 hours and hybridized overnight in a buffer containing 50% formamide, 5×SSC, 5×Denhardt's solution, 0.05 M phosphate, 5 mM EDTA, 0.1% SDS and 250 μg/ml fish sperm. External probes located 5′ and 3′ of the Hsd17b7 gene were radioactively labeled using the REDIPRIME II Random Primer Labeling Kit (GE Healthcare, UK). The blots were exposed overnight at −80° C. to KODAK BIOMAX MS film (Sigma, St. Louis, Mo.) with an intensifier screen. The Southern blots were visualized by autoradiography.

Genotyping of Mouse Embryos via PCR. Offspring were genotyped by PCR for HSD17B7^{+/-} mice using genomic DNA extracted from mice tails (Direct PCR lysis buffer; Viagen Biotech, Los Angeles, Calif.). To identify the wild-type allele, primers to exon 2 were used: P1, 5′-CAC ATA ATG TTT GGG TAT TTT CTA-3′ (SEQ ID NO:3); and P2, 5′-AGC AAT CTA TGA CAA AGA GAC ATA-3′ (SEQ ID NO:4). Two sets of primers were used to identify the HSD17B7 recombinant allele: set 1, (a) 5′-TGT ATG AGC GTT TGG TAG TTA CCA-3′ (SEQ ID NO:5) and (b) 5′-AGT TCT TCT GAG GGG ATC AAT TCA-3′ (SEQ ID NO:6); and set 2, (c) 5′-ATC TTT TCC CTT TGT TTC TGG TCA-3′ (SEQ ID NO:7) and (d) 5′-TCG ACG AAG CTA ATT CAT AAC TTC-3′ (SEQ ID NO:8). See third diagram in FIG. 1. PCR was performed in 50 mM Tris (pH 9.0), 16 mM (NH₄)₂SO₄, 1.75 mM MgCl₂, 1 mM dNTPs and 40 U KOD Taq polymerase (NOVAGEN, Gibbstown, N.J.). The cycling parameters were 94° C. for 15 seconds, 63° C. for 30 seconds and 72° C. for 2.5 minutes for 35 cycles. Each PCR product was cloned in pGEM-Teasy (PROMEGA, Madison, Wis.) vector and each sequence was confirmed as part of mouse HSD17B7 gene by BLAST analysis. For embryos of mid-gestational stage, genomic DNA was isolated from the yolk sac. Recombinant primers to give an approximately 2 kb piece for genotyping of yolk sac were: 5′-GGG ATA ACG ATT ATA ACA GC-3′ (SEQ ID NO:9) and 5′-GAT GTG CAA CTC ACA TAA AG-3′ (SEQ ID NO:10). PCR was performed in 1×PCR buffer, 1.75 mM MgSO₄, 1 mM dNTPs, 20 pmol primers, and 10 U KOD Taq polymerase (NOVAGEN, Gibbstown, N.J.). The cycling parameters were 94° C. for 315 seconds, 61° C. for 30 seconds and 72° C. for 1.75 minutes for 35 cycles. Each PCR product was cloned in pGEM-Teasy (PROMEGA, Madison, Wis.) vector and each sequence was confirmed as part of mouse HSD17B7 gene by BLAST analysis.

Whole Mount In Situ Hybridization. In situ hybridization probes were generated by PCR using cDNA of ovaries at day 15 of pregnancy as a template (Marijanovic, et al. (2003) supra). PCR product was cloned in pGEMT-easy vector (PROMEGA, Madison, Wis.), sequenced and confirmed to be part of the mouse HSD17B7 gene (base pairs 213 to 806; GENBANK Accession No. NM_{—}010476). HSD17B7 cDNA was digested with either SpeI/Sp6 (antisense) or NcoI/T7 (sense) and subjected to in vitro transcription (Roche Diagnostics Corporation, Indianapolis, Ind.) to generate cRNA probes. Antisense probes used in this study were specific for HSD17B7 and sense probes served as negative control. In situ hybridization was performed essentially as described (Wilkinson & Nieto (1993) Methods Enzymol. 225:361-373). Briefly, embryos were fixed in 4% paraformaldehyde (PFA) at 4° C., dehydrated in a graded methanol series and stored at −20° C. in 100% methanol until in situ hybridization was performed. Embryos were then rehydrated, bleached in methanol/H₂O₂ (4:1) for 1 hour, washed in PEST, treated with proteinase K (4-5 minutes E9.5), post-fixed in 4% PFA/0.2% glutaraldehyde, and hybridized with digoxigenin-labeled cRNA probes. Hybridized cRNA probes were detected with sheep anti-DIG Alkaline Phosphatase-conjugated FAB antibody (Roche Diagnostics Corporation, Indianapolis, Ind.). After BCIP/NBT (Roche Diagnostics Corporation, Indianapolis, Ind.) reaction to detect signal, embryos were dehydrated through a graded methanol series to develop the purple colored precipitate, rehydrated and cleared in 50% glycerol prior to imaging.

Immunohistochemistry. For histological analysis, post-partum ovaries were dissected and fixed in Bouin's fixative (SIGMA, St. Louis, Mo.). Concepti from wild-type timed matings were removed from the uteri and fixed in their surrounding membranes in 4% PFA. Both post-partum ovaries and concepti were serially sectioned (5 μm) and stained with Hematoxylin and Eosin using standard procedures. To examine whether post-partum ovaries and wild-type concepti expressed PRAP/17βHSD-7, sections were incubated overnight at 4° C. with a primary polyclonal antibody PRAP/17βHSD-7 (1:150 dilution) (Parmer, et al. (1992) supra) then incubated with a secondary biotinylated goat anti-rabbit IgG according to manufacturer's instructions (VECTASTAIN ABC kit, Vector Laboratories, Burlingame, Calif.). Peroxidase activity was developed with Nova Red solution (Vector Laboratories, Burlingame, Calif.) and sections were counterstained with hematoxylin (Vector Laboratories, Burlingame, Calif.).

### Example 2

### Targeted Deletion of the Mouse HSD17B7 Gene

To investigate the function of HSD17B7 in mammalian development, the Hsd17b7 coding sequence was disrupted by homologous recombination. The mouse HSD17B7 protein is encoded by eleven exons. To disrupt the NADH+/catalytic domain of HSD17B7, a neomycin resistance (neo) gene cassette was inserted into exons 1 through 4 (FIG. 1). Out of approximately 300 colonies resistant to both G418 and ganciclovir, ten clones were expanded and screened for homologous recombination. Two probes, probe A and B, were used for Southern blot analysis of ES cells to identify the recombinant allele at 15 kb and wild-type allele at 10 kb. PCR primers for the flanking region outside both the 5′ and 3′ homologous recombination sites gave PCR products of either 5.6 kb or 5.4 kb corresponding to the recombinant allele. Of these ES cell clones, one clone had a legitimate homologous recombination event in one allele of the Hsd17b7 gene as confirmed by both Southern blot and PCR. To generate chimeric mice, the HSD17B7 homologous recombinant ES cell clones were injected into blastocysts from C57BL/6 mice and the injected blastocysts were transplanted to pseudopregnant females. One of the clones transmitted to the germline resulted in 16 male chimeric mice, which were subsequently backcrossed to the C57BL/6 background. The F1 mice were genotyped by Southern blot and PCR.

### Example 3

### HSD17B7^{-/-} Lethality in Utero

Intercross of HSD17B7^{+/-} mice gave offspring that were either wild-type (HSD17B7^{+/+}) or heterozygous (HSD17B7^{+/-}). In total, of 314 offspring analyzed by Southern blot and PCR, 213 (68%) were HSD17B7^{+/-} and 101 (32%) were HSD17B7^{+/+}. Unexpectedly, however, homozygous null (HSD17B7^{-/-}) live-born offspring (Table 1) were not obtained.

TABLE 1

| | Total | +/+ | +/− | −/− |
|---|---|---|---|---|
| Chimera | 6 | | | |
| Chimera × Wild-Type | 48 | 23 | 25 | 0 |
| F₁ (+/− × +/−) | 105 | 36 | 69 | 0 |
| F₂ (+/− × +/−) | 129 | 41 | 88 | 0 |
| F₃ (+/− × +/−) | 80 | 24 | 56 | 0 |

It was determined whether the deletion of one allele was sufficient to silence the expression of HSD17B7 in HSD17B7^{+/-} mice. Because this enzyme is highly expressed in the corpus luteum, post-partum ovaries were isolated from either wild-type or HSD17B7 females and subjected to immunohistochemistry using a highly specific antibody (Parmer, et al. (1992) supra). This analysis indicated that HSD17B7 was expressed similarly in the corpora lutea of the heterozygous and wild-type mice. Furthermore, HSD17B7^{+/-} mice appeared entirely normal in their development and gross anatomy. The females cycled normally and both male and female were fertile with normal litter size. This indicated that one allele was sufficient for normal expression of HSD17B7 and for maintenance of pregnancy. The absence of newborn homozygous null mice indicated that targeted disruption of HSD17B7 may either prevent implantation or affect fetal survival during embryogenesis.

To determine whether the absence of HSD17B7 homozygous null mice was due to a defect in implantation or embryonic lethality in utero, HSD17B7^{+/-} mice were intercrossed and the day a vaginal plug was found was considered day 0.5 (E 0.5) of pregnancy. On day 10.5 of pregnancy, the concepti were isolated and embryos were dissected out from the yolk sac. The yolk sac was used for genotyping by both Southern blot and PCR. HSD17B7^{-/-} fetuses were found until E11.5. From this stage severe fetal disintegration and numerous resorption sites were detected. No resorptions sites were found earlier of E10.5 and no −/− embryos were recovered after E11.5. At E8.5 and E9.5, HSD17B7^{-/-} fetuses were present in Mendelian ratio. Of the ten E10.5 litters examined, 22 HSD17B7^{+/+}, 52 HSD17B7^{+/-} and 12 HSD17B7^{-/-} embryos were recovered. These results clearly indicated that implantation was unaffected by the deletion of HSD17B7 in embryos and that lethality occurred during development (Table 2).

TABLE 2

| Number of | | | | | |
|---|---|---|---|---|---|
| Heterozygous | Embryonic | Wild-Type | Heterozygous | Null | Resorbed |
| Mating | Development | Embryos | Embryos | Embryos | Embryos |
| 40 | Newborn | 101 | 213 | 0 | 0 |
| 5 | E11.5 | 10 | 20 | 4 | 7 |
| 10 | E10.5 | 22 | 52 | 12 | 0 |
| 4 | E9.5 | 6 | 20 | 8 | 0 |
| 4 | E8.5 | 11 | 15 | 6 | 0 |

### Example 4

### Timing of Fetal Lethality in Utero

To examine the exact time of embryonic death, embryos were dissected from timed pregnant HSD17B7+/− mice. Fetuses were isolated at different days of gestation between E7.5 and E18.5 and checked for survival. PCR analyses of the yolk sac confirmed the existence of the HSD17B7 mutation and loss of the HSD17B7 wild-type allele. At E8.5 and E9.5 −/− (Table 2) null embryos were recovered at Mendelian ratios with similar size to wild-type littermates and no signs of resorption were noted. Well-established AP (antero-posterior) axial structures and somites were dissected and a beating heart was present in embryos. The earliest recognizable defects were detected in E10.5 HSD17B7^{-/-} embryos. The fetuses appeared distressed with no detectable blood in the heart and no heart beat. Lack of vascularization was also seen in both the fetus and the yolk sac indicating that the defect occurred between E9.5 and E10.5, a stage of major embryonic development. On E11.5, significant embryo resorption was observed with severe fetal disintegration. When E10.5 embryos were isolated, the null embryos were found to be smaller in size than their wild-type littermates.

Pathology report of the HSD17B7^{-/-} whole embryos indicated that the major defects occurred in the development of the nervous system. In normal brain development, there were major cell migration, cell differentiation and invaginations specifically in the region of forebrain, midbrain and hindbrain. This stage of embryo development is normally accompanied by an increase in the volume of the forebrain and the fourth ventricle in the hindbrain region. A drastic reduction in size of these regions was found in the null embryos as compared to the wild-type and heterozygous littermates. Histological studies of the brain region of wild-type and null HSD17B7 embryos at E10.5 confirmed the pathological studies and showed a marked underdevelopment and reduced size and volume in the forebrain and midbrain. Furthermore, the boundaries between each of these regions were very poorly defined in null embryo as compared to the wild-type. In addition, null embryos also had a heart defect with abnormal accumulation of fluid in the pericardial cavity.

### Example 5

### Placental-Specific Expression of HSD17B7 Gene

Since pathological analysis of the HSD17B7^{-/-} fetuses revealed major defects in brain and heart development, the tissue-specific expression of HSD17B7 enzyme was examined in the fetus. Whole mount in situ hybridization analysis was conducted on wild-type embryos using a HSD17B7-specific probe. This analysis indicated HSD17B7 expression not only in the brain and heart, but in the eye and ear as well. The localization of HSD17B7 in the placenta was also examined by immunohistochemistry. This enzyme was found to be expressed in the ectoplacental cone and in the spongiotrophoblast, confirming a previous report (Nokelainen, et al. (2000) Endocrinology 141:772-8). However, HSD17B7 was also clearly detected in the giant cells. Moreover, HSD17B7 was expressed in the maternal decidua, however, the expression of this enzyme in the mother could not rescue the embryo.

### Example 6

### Role of the Cholesterol Biosynthetic Activity of HSD17B7 in Fetal Development

To demonstrate the role of HSD17B7 in pregnancy and/or in fetal development, the HSD17B7 coding sequence was disrupted by homologous recombination. Since this enzyme is abundantly expressed in the corpus luteum, where it converts estrone to the potent estradiol, and because estradiol was shown to act locally to stimulate the growth, vascularization and progesterone production of the corpus luteum (Stocco, et al. (2006) supra; Risk & Gibori (2001) supra; Bowen-Shauver & Gibori (2004) supra), severe defects were expected in the development and steroidogenic capacity of this gland leading to abortion. In addition, the finding that this enzyme was not expressed in the granulosa cells cast doubt on the conventional theory that the large luteal cells originates from the follicular granulosa cells (Stocco, et al. (2006) supra). Instead, it was contemplated that estradiol formed in the large luteal cells was responsible for the hypertrophy and increase in size of these cells. Therefore it was expected that the large luteal cells would remain the same size as the small luteal cells in the HSD17B7 null mice. However, unexpectedly, the deletion of Hsd17b7 gene led to embryonic lethality after day 9.5 of pregnancy.

The detection of this enzyme in the fetal forebrain, midbrain and hindbrain regions of wild-type embryos and its absence in null mice strongly indicated that HSD17B7 plays a key role in the normal formation of the brain in utero. Estradiol was shown to be required for normal brain maturation (Bakker, et al. (2003) Ann. NY Acad. Sci. 1007:251-262; Bakker & Baum (2008) Front Neuroendocrinol. 29:1-16). A crucial role for estradiol/ERα has also been found during the initial phases of differentiation of brain cells that sequentially involves both glia and neurons (Merlo, et al. (2007) Mol. Cell Neurosci. 34:562-570). Estradiol was shown to promote proliferation of embryonic cortical progenitor cells in vitro whereas blockade of estrogen receptors in utero decreases proliferation (Martínez-Cerdeño, et al. (2006) Eur. J. Neurosci. 24:3475-88). CYP19, the enzyme responsible for the formation of estrone that serves as substrate for HSD17B7, was present in the embryonic neocortex. These findings indicate a functional role for estradiol as a proliferative agent during critical stages of cerebral cortex development. Since HSD17B7 converts estrone to estradiol and CYP19 is essential for estrone biosynthesis, one would expect to see fetal death in the CYP19 null mice similar to that seen in the HSD17B7 null mice if estradiol was essential for embryo survival. Nevertheless, the finding that female and male mice lacking functional CYP19 are born and grow to adulthood (Fisher, et al. (1998) Proc. Natl. Acad. Sci. USA 95:6965-6970) indicates that estradiol is not essential for fetal brain development and fetal survival. Therefore, the reason of the lethality seen in the mice of this invention appears not to be due to the lack of conversion of estrone to estradiol, but rather due to a different activity of this enzyme. Indeed, it has been reported that in addition to converting estrone to estradiol, HSD17B7 is the last unknown enzyme in mammalian cholesterol biosynthesis and has 3-ketosteroid reductase activity (Breitling, et al. (2001) supra; Marijanovic, et al. (2003) supra). It can convert zymosterone to zymosterol, an important step in cholesterol biosynthesis. Unlike nonneuronal cells, the viability of neurons depends on the intracellular cholesterol content (Michikawa & Yanagisawa (1999) J. Neurochem. 72:2278-2285). Originally, it was demonstrated that the ortholog of the yeast 3-ketosteroid reductase, Erg27p, converts zymosterone to zymosterol in vitro. Expression of human and murine Hsd17b7 in an Erg27p-deficient yeast strain and in a HSD17B7 mammalian deficient cells complemented the 3-ketosteroid reductase deficiency of the cells and restored growth in cholesterol-deficient medium (Ohnesorg, et al. (2006) J. Mol. Endocrinol. 37:185-197; Seth, et al. (2006) J. Biotechnol. 121:241-252) further establishing the role of HSD17B7 in the cholesterol biosynthesis pathway and demonstrating that this enzyme is the last undiscovered enzyme in this pathway. Promoter analysis substantiated the finding that HSD17B7 is involved in postsqualene cholesterol biosynthesis in both humans and mice (Ohnesorg & Adamski (2006) Mol. Cell Endocrinol. 248:164-167).

It was originally contemplated that the involvement of HSD17B7 in cholesterol biosynthesis in the corpus luteum was of little importance because the cholesterol used by the ovary originates from circulating lipoprotein (Ferreri, et al. (1992) Endocrinology 131:2059-2064; Khan, et al. (1985) Biol. Reprod. 32:96-104). However, the marked expression of this enzyme in the fetal brain and the fact the viability of neurons depends on the intracellular cholesterol content and not on the intermediate nonsterol isoprenoid products (Michikawa & Yanagisawa (1999) supra) indicated the importance of this activity in brain formation. The demands for cholesterol in the fetus are high, but whereas maternal cholesterol substantially contributes to fetal cholesterol during early pregnancy, fetal cholesterol biosynthesis rather than cholesterol transfer from maternal lipoproteins seems to be the main mechanism for satisfying fetal requirements (Herrera (2000) Eur. J. Clin. Nutr. 54 Suppl 1:S47-S51; Tint, et al. (2006) J. Lipid Res. 47:1535-1541). From E10 the blood-brain barrier is formed and the brain rapidly becomes the source of almost all of its own cholesterol (Herrera (2000) supra; Tint, et al. (2006) supra). Interestingly, E10 is the stage where severe defects in brain formation are most apparent in the HSD17B7 null embryos. In humans, genetic defects in the enzymes of the cholesterol biosynthetic pathway, leads to abnormal brain development in utero causing mental retardation (Fitzky, et al. (1998) Proc. Natl. Acad. Sci. USA 95:8181-8186; Salen, et al. (1996) J. Lipid Res. 37:1169-1180). Similar brain developmental anomalies and embryonic lethality to the Hsd17b7^{-/-} fetuses have also been observed in mice with deletion of other proteins involved in cholesterol synthesis (Tozawa, et al. (1999) J. Biol. Chem. 274:30843-30848; Ohashi, et al. (2003) J. Biol. Chem. 278:42936-42941; Saher, et al. (2005) Nat. Neurosci. 8:468-475). The most studied enzyme is squalene synthase that catalyzes the first committed step in cholesterol biosynthesis. The complete knockout displays an early embryonic lethal phenotype similar to that of HSD17B7 (Tozawa, et al. (1999) supra). The squalene synthase null fetuses have undeveloped brain and also die after day 9.5 at a time when the brain barrier is formed and the brain becomes dependent on its own synthesis of cholesterol. These fetuses are also smaller the day they die.

Subsequent investigations generated conditional mouse lines, with cell-type deletion of squalene synthase and thus cholesterol production in specific cells in the brain. When squalene synthase was eliminated only in myelinating glia cells (Saher, et al. (2005) supra; Fünfschilling, et al. (2007) BMC Neurosci. 8:1), mutant animals were born but showed severe hypomyelination and a third of the new born died. When squalene synthase was deleted embryonically in neuronal and glial precursors, no pups were born (Saher, et al. (2005) supra; Fünfschilling, et al. (2007) supra), highlighting the importance of brain cholesterol synthesis during development for fetal survival. The deletion of Hsd17b7, the last enzyme in the cholesterol biosynthetic pathway to be discovered, further establishes the importance of cholesterol synthesis in brain development.

In addition to the expression of HSD17B7 in the brain, this enzyme was also found in the heart. Interestingly, the pathological analysis of the Hsd17b^{-/-} fetuses indicates a defect in the heart with pericardial effusion. Whether this pathology is responsible for the lack of vascularization found in the E10.5 fetus is to be investigated. Deletion of HSD17B7 is not expected to deplete heart cholesterol since the heart receives its cholesterol from the maternal circulation. This defect may be due to accumulation of the precursors of zymosterol or to a yet undefined HSD17B7 activity involved in proper vascular development. Generation of HSD17B7 conditional knockout will unravel the importance of this enzyme in the brain verses vascular system in fetal survival.

HSD17B7 is also expressed in the trophoblast, yet deletion of this enzyme did not affect the apparent morphology of the placenta most likely due to the fact that the placenta obtains its cholesterol from circulating lipoprotein (Gibori, et al. (1988) Rec. Prog. Hormone Res. 44: 377-429). In addition, because of the absence of CYP19 (Gibori, et al. (1988) supra; Warshaw, et al. (1986) Endocrinology 119:2642-2648), the placenta is unable to produce estrone that is converted to estradiol by HSD17B7 and estradiol is generated during pregnancy by the maternal ovary. This further substantiates the specific and important role of the cholesterol biosynthetic activity of HSD17B7 in fetal development.

What is claimed is:

1. A knockout transgenic non-human animal whose genome comprises a deletion in the endogenous 17β Hydroxysteroid Dehydrogenases Type 7 gene, wherein said animal exhibits one or more signs or symptoms of neuronal tube defects indicative of anencephaly.

2. A cell line derived from the knockout transgenic non-human animal of claim 1.

3. A method of screening for a prophylactic or therapeutic agent for anencephaly comprising administering a test agent to the animal of claim 1 and evaluating the effect of the test agent on anencephaly.

4. A therapeutic agent identified by the method of claim 3.

5. A method of diagnosing anencephaly comprising determining whether a subject has a mutation, insertion or deletion in the gene encoding 17β Hydroxysteroid Dehydrogenases Type 7 (HSD17B7) or has a decrease in the expression or activity of HSD17B7 as compared to a control, wherein said mutation, insertion deletion, or decrease in expression or activity of HSD17B7 is indicative of anencephaly.

6. A method for preventing or treating anencephaly comprising administering to a subject in need of treatment an effective amount of an agent of claim 4 thereby preventing or treating anencephaly.

7. The method of claim 6, wherein the agent is 17β Hydroxysteroid Dehydrogenases Type 7 protein or a nucleic acid encoding the same.


SEQUENCE LISTING (10 sequences)

SEQ ID NO: 1 - protein, 334 aa, Mus musculus
MRKVVLITGASSGIGLALCGRLLAEDDDLHLCLACRNLSKARAVRDTLLASHPSAEVSIVQMDVSSLQSVVRGAEEVKQKFQRLDYLYLNAGILPNPQFNLKAFFCGIFSRNVIHMFTTAEGILTQNDSVTADGLQEVFETNLFGHFILIRELEPLLCHADNPSQLIWTSSRNAKKANFSLEDIQHSKGPEPYSSSKYATDLLNVALNRNFNQKGLYSSVMCPGVVMTNMTYGILPPFIWTLLLPIMWLLRFFVNALTVTPYNGAEALVWLFHQKPESLNPLTKYASATSGFGTNYVTGQKMDIDEDTAEKFYEVLLELEKRVRTTVQKSDHPS

SEQ ID NO: 2 - protein, 334 aa, Rattus norvegicus
MRKVVLITGASSGIGLALCGRLLAEDDDLHLCLACRNLSKAGAVRDALLASHPSAEVSIVQMDVSNLQSVVRGAEEVKRRFQRLDYLYLNAGIMPNPQLNLKAFFCGIFSRNVIHMFSTAEGLLTQNDKITADGFQEVFETNLFGHFILIRELEPLLCHSDNPSQLIWTSSRNAKKSNFSLEDIQHAKGQEPYSSSKYATDLLNVALNRNFNQKGLYSSVTCPGVVMTNLTYGILPPFVWTLLLPVIWLLRFFAHAFTVTPYNGAEALVWLFHQKPESLNPLTKYLSGTTGLGTNYVKGQKMDVDEDTAEKFYKTLLELEKQVRITIQKSDHHS

SEQ ID NO: 3 - DNA, 24 bp, Artificial sequence
  Synthetic oligonucleotide
cacataatgtttgggtattttcta

SEQ ID NO: 4 - DNA, 24 bp, Artificial sequence
  Synthetic oligonucleotide
agcaatctatgacaaagagacata

SEQ ID NO: 5 - DNA, 24 bp, Artificial sequence
  Synthetic oligonucleotide
tgtatgagcgtttggtagttacca

SEQ ID NO: 6 - DNA, 24 bp, Artificial Sequence
  Synthetic oligonucleotide
agttcttctgaggggatcaattca

SEQ ID NO: 7 - DNA, 24 bp, Artificial Sequence
  Synthetic oligonucleotide
atcttttccctttgtttctggtca

SEQ ID NO: 8 - DNA, 24 bp, Artificial Sequence
  Synthetic oligonucleotide
tcgacgaagctaattcataacttc

SEQ ID NO: 9 - DNA, 20 bp, Artificial Sequence
  Synthetic oligonucleotide
gggataacgattataacagc

SEQ ID NO: 10 - DNA, 20 bp, Artificial Sequence
  Synthetic oligonucleotide
gatgtgcaactcacataaag